(19) United States
(12) Patent Application Publication
(10) Pub. No.: US 2016/0306935 A1
(43) Pub. Date: Oct. 20, 2016

(54) METHODS AND SYSTEMS FOR PREDICTING A HEALTH CONDITION OF A HUMAN SUBJECT

(71) Applicant: XEROX CORPORATION, Norwalk, CT (US)

(72) Inventors: Vaibhav Rajan, Bangalore (IN); Sakyajit Bhattacharya, Bangalore (IN)

(21) Appl. No.: 14/687,128

(22) Filed: Apr. 15, 2015

(51) Int. Cl. G06F 19/00; A61B 5/08; A61B 5/021; A61B 5/00; A61B 5/145

(52) U.S. Cl. CPC G06F 19/345; A61B 5/7264; A61B 5/14532; A61B 5/021; A61B 5/082

(57) ABSTRACT

Disclosed are embodiments of methods and systems for predicting a health condition of a first human subject. The method comprises extracting a historical data including physiological parameters of one or more second human subjects. A latent variable is determined based on an inverse cumulative distribution of a transformed historical data, determined by ranking of the historical data. Further, one or more parameters of a first distribution, deterministic of health conditions in the historical data, are determined based on the latent variable. For each physiological parameter, a random variable is sampled from a second distribution of the physiological parameter based on the one or more parameters. Further, based on the random variable, the latent variable is updated. Thereafter, the one or more parameters are re-estimated based on the updated latent variable. Based on the first distribution a classifier is trained to predict the health condition of the first human subject.

### TECHNICAL FIELD

The presently disclosed embodiments are related, in general, to healthcare. More particularly, the presently disclosed embodiments are related to methods and systems for predicting a health condition of a human subject.

### BACKGROUND

Various industries, including the healthcare industry, may maintain records of the various stakeholders involved with the industry. For example, the healthcare industry may maintain various records of human subjects/patients such as, but not limited to, medical diagnosis records, medical insurance records, hospital data, etc. Thereafter, one or more mathematical models may be used to identify trends and categorize the records into various categories such as health conditions of human subjects/patients, health insurance fraud risks, and so on.

Usually, the records include data in various data types such as numerical data type (e.g., BP measure, heart rate, and blood sugar measure) and categorical data type (e.g., gender). Further, the mathematical models used to analyse the records may only consider the data of numerical data type in the medical records, to identify the trends across the medical records.

### SUMMARY

According to embodiments illustrated herein there is provided a method for predicting a health condition of a first human subject. The method comprises extracting, by one or more processors, a historical data comprising a measure of one or more physiological parameters associated with each of one or more second human subjects. Thereafter, a latent variable is determined based on an inverse cumulative distribution of a transformed historical data. The transformed historical data is determined by ranking of the historical data. Further, one or more parameters of a first distribution, which is deterministic of one or more health conditions in the historical data, are determined based on the latent variable. For each physiological parameter from the one or more physiological parameters, a random variable is sampled from a second distribution of the physiological parameter based on the one or more parameters. Further, for each physiological parameter, the latent variable is updated based on the random variable. Thereafter, the one or more parameters are re-estimated based on the updated latent variable. Further, a classifier is trained based on the first distribution. The one or more processors receive a measure of the one or more physiological parameters associated with the first human subject. Thereafter, the health condition of the first human subject is predicted by utilizing the classifier based on the received measure of the one or more physiological parameters associated with the first human subject.

According to embodiment illustrated herein there is provided a system for a health condition of a first human subject. The system comprising one or more processors configured to extract a historical data comprising a measure of one or more physiological parameters associated with each of one or more second human subjects. Thereafter, a latent variable is determined based on an inverse cumulative distribution of a transformed historical data. The transformed historical data is determined by ranking of the historical data. Further, one or more parameters of a first distribution, which is deterministic of one or more health conditions in the historical data, are determined based on the latent variable. For each physiological parameter from the one or more physiological parameters, a random variable is sampled from a second distribution of the physiological parameter based on the one or more parameters. Further, for each physiological parameter, the latent variable is updated based on the random variable. Thereafter, the one or more parameters are re-estimated based on the updated latent variable. Further, a classifier is trained based on the first distribution. The one or more processors are further configured to receive a measure of the one or more physiological parameters associated with the first human subject. Thereafter, the health condition of the first human subject is predicted by utilizing the classifier based on the received measure of the one or more physiological parameters associated with the first human subject.

According to embodiment illustrated herein there is provided a computer program product for use with a computing device. The computer program product comprising a non-transitory computer readable medium. The non-transitory computer readable medium stores a computer program code for predicting a health condition of a first human subject. The computer program code is executable by one or more processors in the computing device to extract a historical data comprising a measure of one or more physiological parameters associated with each of one or more second human subjects. Thereafter, a latent variable is determined based on an inverse cumulative distribution of a transformed historical data. The transformed historical data is determined by ranking of the historical data. Further, one or more parameters of a first distribution, which is deterministic of one or more health conditions in the historical data, are determined based on the latent variable. For each physiological parameter from the one or more physiological parameters, a random variable is sampled from a second distribution of the physiological parameter based on the one or more parameters. Further, for each physiological parameter, the latent variable is updated based on the random variable. Thereafter, the one or more parameters are re-estimated based on the updated latent variable. Further, a classifier is trained based on the first distribution. The computer program code is further executable by the one or more processors to receive a measure of the one or more physiological parameters associated with the first human subject. Thereafter, the health condition of the first human subject is predicted by utilizing the classifier based on the received measure of the one or more physiological parameters associated with the first human subject.

### BRIEF DESCRIPTION OF DRAWINGS

The accompanying drawings illustrate various embodiments of systems, methods, and other aspects of the disclosure. Any person having ordinary skill in the art will appreciate that the illustrated element boundaries (e.g., boxes, groups of boxes, or other shapes) in the figures represent one example of the boundaries. It may be that in some examples, one element may be designed as multiple elements or that multiple elements may be designed as one element. In some examples, an element shown as an internal component of one element may be implemented as an external component in another, and vice versa. Furthermore, elements may not be drawn to scale.

Various embodiments will hereinafter be described in accordance with the appended drawings, which are provided to illustrate, and not limit, the scope in any manner, wherein similar designations denote similar elements, and in which:

FIG. 1 is a block diagram of a system environment, in which various embodiments can be implemented;

FIG. 2 is a block diagram of a system that is capable of identifying one or more clusters in a multivariate dataset, in accordance with at least one embodiment;

FIGS. 3A and 3B illustrate a flowchart of a method for predicting a health condition of a first human subject, in accordance with at least one embodiment; and

FIGS. 4A and 4B illustrate a flow diagram of a method for predicting a health condition of a first human subject, in accordance with at least one embodiment.

### DETAILED DESCRIPTION

The present disclosure is best understood with reference to the detailed figures and descriptions set forth herein. Various embodiments are discussed below with reference to the figures. However, those skilled in the art will readily appreciate that the detailed descriptions given herein with respect to the figures are simply for explanatory purposes, as the methods and systems may extend beyond the described embodiments. For example, the teachings presented and the needs of a particular application may yield multiple alternate and suitable approaches to implement the functionality of any detail described herein. Therefore, any approach may extend beyond the particular implementation choices in the following embodiments described and shown.

References to “one embodiment,” “at least one embodiment,” “an embodiment,” “one example”, “an example”, “for example” and so on, indicate that the embodiment(s) or example(s) so described may include a particular feature, structure, characteristic, property, element, or limitation, but that not every embodiment or example necessarily includes that particular feature, structure, characteristic, property, element, or limitation. Furthermore, repeated use of the phrase “in an embodiment” does not necessarily refer to the same embodiment.

Definitions: The following terms shall have, for the purposes of this application, the respective meanings set forth below.

A “multivariate dataset” refers to a dataset that includes observations of a p-dimensional variable. For example, “n” observations of p-dimensional variable may constitute a multivariate dataset. For example, a medical record data may include a measure of one or more physiological parameters of one or more patients, where the one or more physiological parameters correspond to the p-dimensions and the one or more patients correspond to n observations. Such medical record data is an example of the multivariate dataset.

A “healthcare dataset” refers to a multivariate dataset that includes data obtained from the healthcare industry. In an embodiment, the healthcare dataset may correspond to a patient record data, hospital data, medical insurance data, diagnostics data, etc. In a scenario, where the healthcare data corresponds to the patient record data, the one or more physiological parameters correspond to the p-dimensional variable, and the number of records in the healthcare data corresponds to the observations.

A “human subject” corresponds to a human being, who may be suffering from a health condition or a disease. In an embodiment, the human subject may correspond to a person who seeks a medical opinion on his/her health condition.

A “Gaussian Mixture Model (GMM)” refers to a mathematical model, which assumes that data values in the multivariate dataset are generated from a mixture of a finite number of Gaussian (or Normal) distributions of unknown parameters. By estimating the parameters of the GMM, one or more clusters may be identified in the multivariate dataset.

A “Gaussian Copula Mixture Model (GCMM)” refers to a mathematical model that is capable of identifying one or more clusters in the multivariate dataset, where data values in each of the one or more clusters are distributed according to a Gaussian Copula distribution. In an embodiment, copula corresponds to a multivariate probability distribution, for which marginal probability of each dimension of the p-dimensional variable is uniformly distributed. In an embodiment, copulas may be used for describing dependence between the dimensions in the dataset. In an embodiment, GCMM may be used for determining trends/identifying clusters in the multivariate dataset, when the data in the multivariate dataset is not normally distributed. A typical Gaussian copula mixture model (GCMM) is represented by the following equation:

$$GCMM = \frac{{\sum }_{g = 1}^{G} {π}_{g} φ ( {z}_{i} \vert {μ}_{g} , {Σ}_{g} )}{{Π}_{j = 1}^{p} {ψ}_{j} ( {z}_{i , j} )} \qquad ( 1 )$$

where,

z_{i,j}: Inverse cumulative distribution of p-dimensional random variable x along j^{th} dimension, such that zᵢ=ψⱼ⁻¹(u_{i,j}) (z_{i,j} is also referred as a latent variable);

u_{i,j}: Cumulative distribution function of p-dimensional random variable x along j^{th} dimension;

p: Number of dimensions of random variable;

π_{g}: Mixing proportion of a cluster g with respect to other clusters in the multivariate dataset;

ψⱼ(z_{i,j}): Marginal density of GMM along j^{th} dimension;

G: Number of clusters in the multivariate dataset;

μ_{g}: Mean of the Gaussian Copula Mixture cluster component g;

Σ_{g}: Covariance matrix of p-dimensional variable x (representative of a covariance of cluster g with other clusters); and

φ(zᵢ|μ_{g}, Σ_{g}): Multivariate Gaussian distribution of a cluster g with mean μ_{g} and variance Σ_{g}.

A “cumulative distribution” refers to a distribution function, that describes the probability that a real-valued random variable X with a given probability distribution will be found at a value less than or equal to x.

An “inverse cumulative distribution” refers to an inverse function of the cumulative distribution of the random variable X.

A “mixing proportion of cluster components” refer to a probability that a data value in the multivariate dataset belongs to different clusters. For example, the multivariate data includes two clusters. A probability that a data value in the multivariate data set belongs to the first cluster is 0.6. Then, the probability that the data value will belong to the second cluster is 0.4. In an embodiment, the sum of probability of the data value in each of the one or more clusters in the dataset is one.

A “latent variable” refers to an intermediate variable that is not obtained from the multivariate dataset. In an embodiment, the latent variable is determined based on one or more parameters of a distribution representing the multivariate dataset. For example, if the distribution representing the multivariate dataset is the Gaussian Copula distribution, the latent variable (denoted as Z) may correspond to the inverse cumulative distribution of the p-dimensional variable (refer to equation 1).

“Probability” shall be broadly construed, to include any calculation of probability; approximation of probability, using any type of input data, regardless of precision or lack of precision; any number, either calculated or predetermined, that simulates a probability; or any method step having an effect of using or finding some data having some relation to a probability.

A “random variable” refers to a variable that may be assigned a value probabilistically or stochastically.

A “classifier” refers to a mathematical model that may be configured to categorize data into one or more categories. In an embodiment, the classifier is trained based on historical data. Examples of the classifier may include, but are not limited to, a Support Vector Machine (SVM), a Logistic Regression, a Bayesian Classifier, a Decision Tree Classifier, a Copula-based Classifier, a K-Nearest Neighbors (KNN) Classifier, or a Random Forest (RF) Classifier.

“Training” refers to a process of updating/tuning a classifier using a historical data such that the classifier is able to predict the one or more categories in the historical data with a greater accuracy.

“Gibbs sampling” refers to a statistical technique that may be used to generate samples from a multivariate distribution. In an embodiment, Gibbs sampling corresponds to a Markov Chain Monte Carlo (MCMC) algorithm for obtaining a sequence of observations from a joint distribution of two or more univariate marginal distributions, when direct sampling from the multivariate distribution may be difficult.

“Expectation Maximization (EM) algorithm” refers to a statistical technique of determining a maximum likelihood estimate of one or more parameters of a distribution, where the distribution depends on unobserved latent variables.

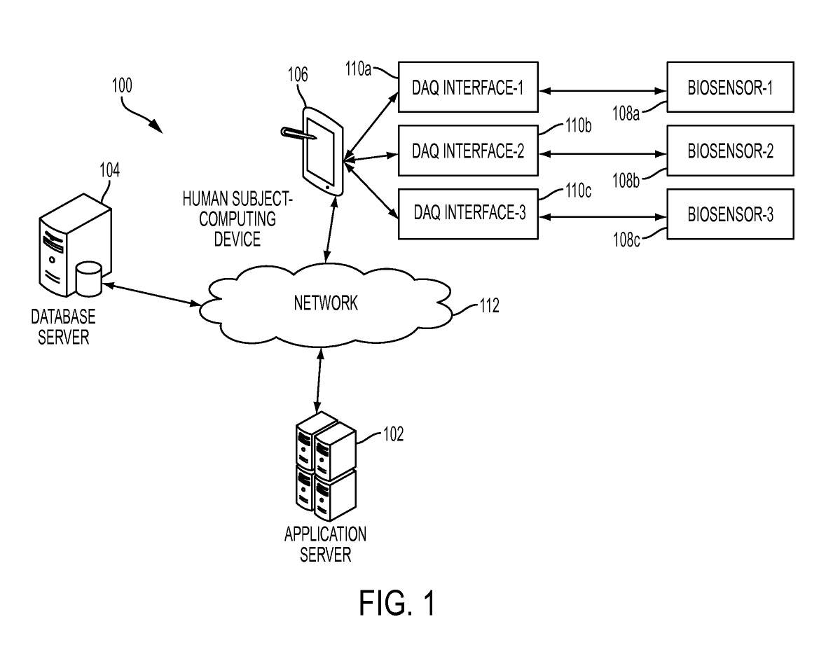

FIG. 1 is a block diagram illustrating a system environment 100 in which various embodiments may be implemented. The system environment 100 includes an application server 102, a database server 104, a human subject-computing device 106, and a network 112.

The application server 102 refers to a computing device including one or more processors and one or more memories. The one or more memories may include computer readable code that is executable by the one or more processors to perform predetermined operation. In an embodiment, the predetermined operation may include predicting a health condition of a first human subject. In an embodiment, the application server 102 may extract a historical data comprising medical records of one or more second human subjects from the database server 104. In an embodiment, a medical record associated with a human subject may include a measure of one or more physiological parameters associated with the human subject. In an embodiment, the application server 102 may apply a rank transformation on the historical data to determine a transformed historical data using an extended rank likelihood technique. Further, the application server 102 may determine a latent variable based on inverse cumulative distribution of the transformed historical data. Thereafter, in an embodiment, the application server 102 may estimate one or more parameters of a first distribution associated with each of the one or more health conditions in the historical data based on the latent variable. In an embodiment, the first distribution may correspond to a GCM distribution and the one or more parameters of the first distribution may include, but are not limited to, a mean, a covariance matrix, and a mixing proportion, of each of one or more cluster components of the first distribution. Thereafter, the application server 102 may re-sample the latent variable for each physiological parameter. To that end, the application server 102 may first determine lower and upper bounds of latent variable for each physiological parameter. Thereafter, the application server 102 may determine a second distribution of the physiological parameter, associated with each of the one or more health conditions in the historical data based on the one or more parameters of the first distribution. Further, the application server 102 may sample a random variable from the second distribution of the physiological parameter. The application server 102 may then update the latent variable based on the sampled random variable. Thereafter, the application server 102 may evaluate a termination condition to determine whether the latent variable is to be re-sampled again. If the termination condition has not been reached, the application server 102 may re-estimate the one or more parameters of the first distribution based on the updated latent variable and then re-sample the latent variable, in a manner similar to that described above. However, if the termination condition has been reached, the application server 102 may determine the first distribution based on the updated latent variable and the one or more parameters associated with the first distribution.

Further, the application server 102 may use the first distribution to identify the one or more health conditions in the historical data. The application server 102 may train a classifier to predict the one or more health conditions in the historical data based on the first distribution. Thereafter, in an embodiment, the application server 102 may receive a measure of the one or more physiological parameters of the first human subject from the human subject-computing device 106 of the first human subject. Alternatively, in a scenario where the one or more physiological parameters of the first human subject are stored on the database server 104, the application server 102 may extract the one or more parameters of the first human subject from the database server 104. In another embodiment, the application server 102 may include one or more biosensors or may be communicatively coupled to the one or more biosensors. The one or more biosensors may determine the measure of the one or more physiological parameters of the first human subject.

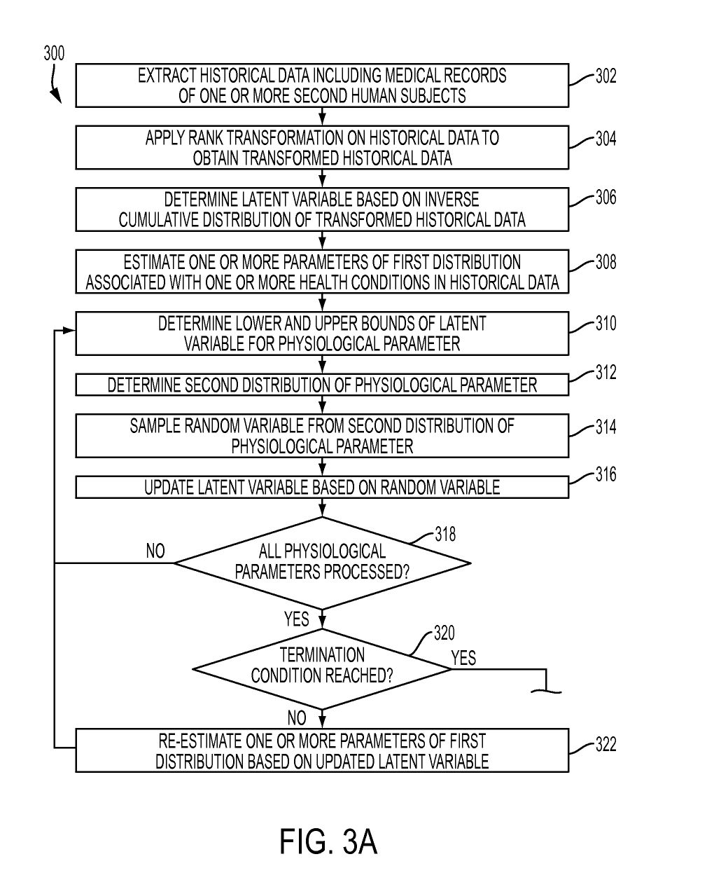

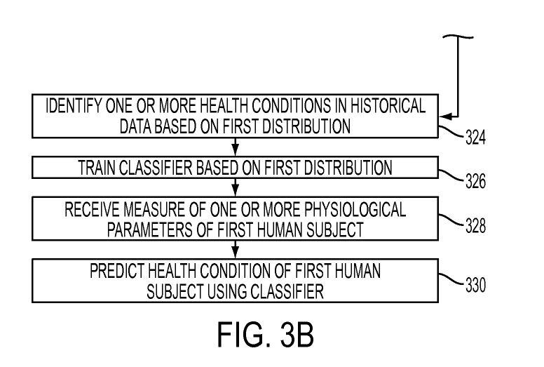

Thereafter, based on the measure of the one or more physiological parameters of the first human subject, the application server 102 may predict the health condition of the first human subject using the classifier. The application server 102 may then display the predicted health condition of the first human subject through a user-interface on the human subject-computing device 106. An embodiment of the prediction of the health condition of the first human subject has been explained further in conjunction with FIGS. 3A and 3B.

The application server 102 may be realized through various types of application servers such as, but not limited to, Java application server, .NET framework application server, and Base 4 application server.

The database server 104 may refer to a computing device, which stores at least the historical data including the medical records of the one or more second human subjects. In an embodiment, the database server 104 may receive the measure of the one or more physiological parameters of each of the one or more second human subjects from the human subject-computing device 106 of the respective second human subject. Thereafter, the database server 104 may store the one or more physiological parameters of the one or more second human subjects as the medical records in the historical data. In addition, in an embodiment, the database server 104 may also store the one or more physiological parameters of the first human subject. In an embodiment, the database server 104 may receive a query from the application server 102 to extract the information stored on the database server 104. The database server 104 may be realized through various technologies such as, but not limited to, Oracle®, IBM DB2®, Microsoft SQL Server®, Microsoft Access®, PostgreSQL®, MySQL® and SQLite®, and the like. In an embodiment, the application server 102 may connect to the database server 104 using one or more protocols such as, but not limited to, Open Database Connectivity (ODBC) protocol and Java Database Connectivity (JDBC) protocol.

A person with ordinary skill in the art would understand that the scope of the disclosure is not limited to the database server 104 as a separate entity. In an embodiment, the functionalities of the database server 104 can be integrated into the application server 102.

The human subject-computing device 106 refers to a computing device used by a human subject (such as the first human subject and the one or more second human subjects). The human subject-computing device 106 may include one or more processors and one or more memories. The one or more memories may include computer readable code that is executable by the one or more processors to perform predetermined operation. In an embodiment, one or more biosensors (e.g., a biosensor-1 108a, a biosensor-2 108b, and a biosensor-3 108c) may be inbuilt within the human subject-computing device 106. Alternatively, the one or more biosensors (e.g., a biosensor-1 108a, a biosensor-2 108b, and a biosensor-3 108c) may be coupled to the human subject-computing device 106 through one or more data acquisition (DAQ) interfaces (e.g., a DAQ interface- 1 110a, a DAQ interface-2 110b, and a DAQ interface-3 110c). For instance, as shown in FIG. 1, the DAQ interface-1 110a may connect the biosensor-1 108a with the human subject-computing device 106. Similarly, the DAQ interface-2 110b may connect the biosensor-2 108b with the human subject-computing device 106, and so on. Examples of the one or more DAQ interfaces, for example, 110a, include but are not limited to, a Universal Serial Bus (USB) Port, a FireWire Port, an IEEE 1394 standard based connector, or any other serial/parallel data interfacing connector known in the art. In another embodiment, the one or more biosensors, e.g., 108a, may be connected to the human subject-computing device 106 through a wireless connection such as, but not limited to, a Bluetooth based connection, a Near Field Communication (NFC) based connection, a Radio Frequency Identification (RFID) based connection, or any other wireless communication protocol.

In an embodiment, the one or more physiological parameters of the human subject may be measured using the one or more biosensors (e.g., a biosensor-1 108a, a biosensor-2 108b, and a biosensor-3 108c). Examples of the one or more physiological parameters include, but are not limited to, a blood glucose level, a blood pressure, an age, a cholesterol level, a heart rate, a breath carbon-dioxide concentration, or a breath oxygen concentration. Thereafter, the human subject-computing device 106 may transmit the measure of the one or more physiological parameters of the human subject to at least one of the application server 102 or the database server 104. In an embodiment, the application server 102 may predict a health condition of the human subject, as described above. Thereafter, the human subject-computing device 106 may display the predicted health condition of the human subject through a user-interface on a display device of the human subject-computing device 106. Based on the predicted health condition of the human subject, the human subject may consult with a medical practitioner.

A person skilled in the art will understand that the scope of the disclosure is not limited to the human subject-computing device 106 being used by the human subject. In an embodiment, the human subject-computing device 106 may be used by a medical practitioner. In such a scenario, when a human subject visits the medical practitioner for a consultation, the medical practitioner may use the human subject-computing device 106 to measure the one or more physiological parameters of the human subject. Thereafter, the human subject-computing device 106 may transmit the one or more physiological parameters of the human subject to at least one of the application server 102 or the database server 104. The application server 102 may predict a health condition of the human subject, as described above. Thereafter, the human subject-computing device 106 may display the predicted health condition of the human subject through the user-interface on a display device of the human subject-computing device 106. Based on the predicted health condition of the human subject, the medical practitioner may recommend a treatment course including one or more medicines, one or more clinical/pathological tests, or one or more diet plans to the human subject.

The human subject-computing device 106 may include a variety of computing devices such as, but not limited to, a laptop, a personal digital assistant (PDA), a tablet computer, a smartphone, a phablet, and the like.

A person skilled in the art will understand that the scope of the disclosure is not limited to the human subject-computing device 106 and the application server 102 as separate entities. In an embodiment, the application server 102 may be realized as an application hosted on or running on the human subject-computing device 106 without departing from the spirit of the disclosure.

The network 112 corresponds to a medium through which content and messages flow between various devices of the system environment 100 (e.g., the application server 102, the database server 104, and the human subject-computing device 106). Examples of the network 112 may include, but are not limited to, a Wireless Fidelity (Wi-Fi) network, a Wireless Area Network (WAN), a Local Area Network (LAN), or a Metropolitan Area Network (MAN). Various devices in the system environment 100 can connect to the network 112 in accordance with various wired and wireless communication protocols such as Transmission Control Protocol and Internet Protocol (TCP/IP), User Datagram Protocol (UDP), and 2G, 3G, or 4G communication protocols.

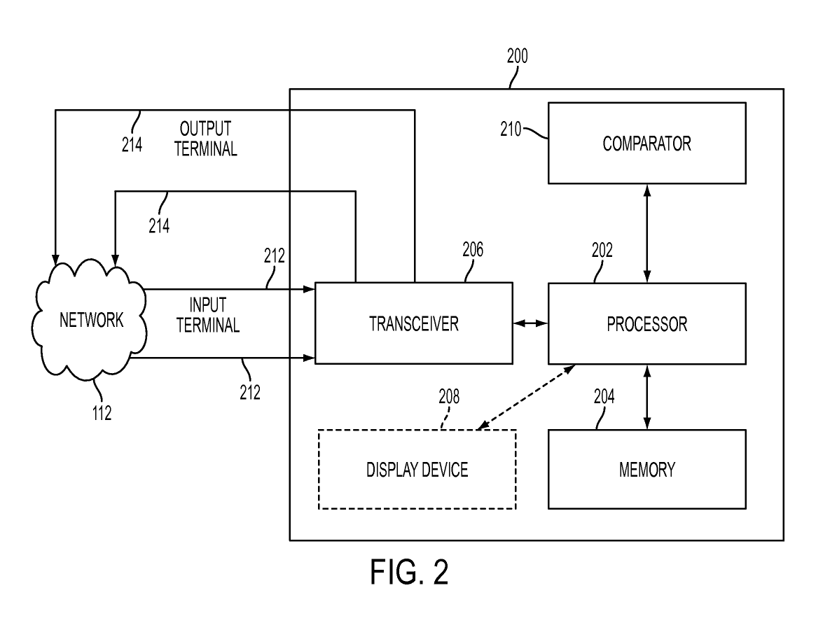

FIG. 2 is a block diagram of a system 200 that is capable of identifying one or more clusters in a multivariate dataset, in accordance with at least one embodiment. In an embodiment, the system 200 may correspond to the application server 102 or the human subject-computing device 106. For the purpose of ongoing description, the system 200 is considered the application server 102. However, the scope of the disclosure should not be limited to the system 200 as the application server 102. The system 200 may also be realized as the human subject-computing device 106, without departing from the spirit of the disclosure.

The system 200 includes a processor 202, a memory 204, a transceiver 206, a display 208, and a comparator 210. The processor 202 is coupled to the memory 204 and the transceiver 206. The transceiver 206 is coupled to a network 112 through an input terminal 212 and an output terminal 214.

The processor 202 includes suitable logic, circuitry, and interfaces and is configured to execute one or more instructions stored in the memory 204 to perform predetermined operations on the computing device 100. The memory 204 may be configured to store the one or more instructions. The processor 202 may be implemented using one or more processor technologies known in the art. Examples of the processor 202 include, but are not limited to, an X86 processor, a RISC processor, an ASIC processor, a CISC processor, or any other processor.

The memory 204 stores a set of instructions and data. Some of the commonly known memory implementations include, but are not limited to, a RAM, a read-only memory (ROM), a hard disk drive (HDD), and a secure digital (SD) card. Further, the memory 204 includes the one or more instructions that are executable by the processor 202 to perform specific operations. It is apparent to a person having ordinary skill in the art that the one or more instructions stored in the memory 204 enable the hardware of the computing device 100 to perform the predetermined operations.

The transceiver 206 transmits and receives messages and data to/from one or more computing devices connected to the computing device 100 over the network 112. Examples of the network 112 may include, but are not limited to, a Wireless Fidelity (Wi-Fi) network, a Wireless Area Network (WAN), a Local Area Network (LAN), or a Metropolitan Area Network (MAN). In an embodiment, the transceiver 206 is coupled to the network 112 through the input terminal 212 and the output terminal 214, through which the transceiver 206 may receive and transmit data/messages respectively. Examples of the transceiver 206 may include, but are not limited to, an antenna, an Ethernet port, a USB port, or any other port that can be configured to receive and transmit data. The transceiver 206 transmits and receives data/messages in accordance with the various communication protocols such as, TCP/IP, UDP, and 2G, 3G, or 4G communication protocols.

The display 208 facilitates a user of the computing device 100 to view information presented on the computing device 100. For example, the user may view a multivariate dataset and one or more clusters identified in the multivariate dataset on the display 208. The display 208 may be realized through several known technologies, such as Cathode Ray Tube (CRT) based display, Liquid Crystal Display (LCD), Light Emitting Diode (LED) based display, Organic LED based display, and Retina display® technology. In an embodiment, the display 208 can be a touch screen that is operable to receive a user-input.

The comparator 210 is configured to compare at least two input signals to generate an output signal. In an embodiment, the output signal may correspond to either “1” or “0.” In an embodiment, the comparator 210 may generate output “1” if the value of a first signal (from the at least two signals) is greater than the value of a second signal (from the at least two signals). Similarly, the comparator 210 may generate an output “0” if the value of the first signal is less than the value of the second signal. In an embodiment, the comparator 210 may be realized through either software technologies or hardware technologies known in the art. Though, the comparator 210 is depicted as independent from the processor 202 in FIG. 1, a person skilled in the art would appreciate that the comparator 210 may be implemented within the processor 202 without departing from the scope of the disclosure.

FIGS. 3A and 3B illustrate a flowchart 300 of a method for predicting a health condition of a first human subject, in accordance with at least one embodiment. The flowchart 300 has been described in conjunction with FIG. 1 and FIG. 2.

At step 302, a historical data including medical records of one or more second human subjects is extracted. In an embodiment, the processor 202 is configured to extract the historical data from the database server 104. In a scenario where the historical data is stored in the memory 204, the processor 202 may extract the historical data from the memory 204. In an embodiment, the historical data may correspond to a multivariate healthcare dataset, which includes a measure of one or more physiological parameters of each of the one or more second human subjects. Examples of the one or more physiological parameters include, but are not limited to, a blood glucose level, a blood pressure, an age, a cholesterol level, a heart rate, a breath carbon-dioxide concentration, and a breath oxygen concentration. In another embodiment, the processor 202 may receive the measure of the one or more physiological parameters of each of the one or more second human subjects from the human subject-computing device 106 of the respective second human subjects. The processor 202 may store the information pertaining to the one or more physiological parameters of the one or more second human subjects as the historical data in the memory 204 or in the database server 104. In an embodiment, the historical data may correspond to a p-dimensional multivariate dataset. The one or more physiological parameters may correspond to a p-dimensional variable. Thus, each physiological parameter may correspond to a different dimension in the p-dimensional multivariate dataset corresponding to the historical data. Further, each medical record in the historical data may correspond to an observation in the p-dimensional multivariate dataset corresponding to the historical data.

A person having ordinary skill in the art would understand that the scope of disclosure is not limited to the aforementioned physiological parameters. In an embodiment, various other physiological parameters may be used without departing from the spirit of the disclosure.

Further, in an embodiment, the processor 202 may receive a user-input pertaining to a number of the one or more health conditions (denoted by G clusters) in the multivariate dataset corresponding to the historical data.

At step 304, a rank transformation is applied on the historical data to obtain a transformed historical data. In an embodiment, the processor 202 is configured to obtain the transformed historical data by applying the rank transformation on the historical data using an extended rank likelihood technique. To generate the transformed historical data, the processor 202 determines ranks of the individual observations in each of the p-dimensions in the historical data. In an embodiment, the processor 202 may assign a rank 1 to an observation having the lowest value in a particular dimension. Further, the processor 202 may assign a rank 2 to an observation having the next highest observation in that dimension, and so on till a rank N to an observation having the highest value in the particular dimension in the historical data. Thereafter, in an embodiment, the processor 202 may divide each rank by N so that the final values of the ranks of the observations lie between 0 and 1. The final values of the ranks of the observations, which lie between 0 and 1, may correspond to the transformed historical data. For example, the historical data includes five observations. The values of the five observations for a particular dimension may include the values 0.1, 5.6, 3.1, 0.8, and 2.2. The processor 202 may assign the ranks 1, 5, 4, 2, and 3 to the observations. Further, the processor 202 may determine the final values of the ranks, and hence the transformed historical data as 0.2, 1, 0.8, 0.4, and 0.6 (i.e., by dividing the ranks by 5).

In case of a GCM distribution, in an embodiment, without knowledge of marginals F, of the copula associated with the GCM distribution, and without observing values of a latent variable Z (refer equation 1), based on the observations in the historical data (i.e., y_{i,j}), the processor 202 may determine that the values of the latent variable Z may lie in a set D represented as under:

D={Z∈R^{n×p}:max{zₖⱼ:yₖⱼ<yᵢⱼ}<zᵢⱼ<min{zₖⱼ:yᵢⱼ<yₖⱼ}} (2)

where,

D: a set representing a range of values within which the latent variable Z is constrained based on observations in the historical data (i.e., y_{i,j );}

z_{i,j}: the value of the latent variable for the i^{th} observation of the j^{th} physiological parameter in the historical data;

y_{i,j}: i^{th} observation of the j^{th} physiological parameter in the historical data;

n: number of observations in the historical data; and

p: number of physiological parameters in the historical data.

Thereafter, the processor 202 may determine a rank likelihood as a probability of the latent variable Z lying in the set D using the following equation:

P(Z∈D|Θ,F₁, F₂, . . . Fₚ)=∫_{D}P(Z|Θ)dZ=P(Z∈D|Θ) (3)

where,

Θ: the one or more parameters of the GCM distribution (a first distribution);

F₁, F₂, . . . Fₚ: marginals of the copula associated with the GCM distribution (the first distribution); and

P(Z∈D|Θ): the rank likelihood of the latent variable Z.

A person skilled in the art would appreciate that the historical data may include data of various data types such as, but not limited to, a numerical data type or a categorical data type. However, in an embodiment, the transformed historical data may include only the ranks. Further, the transformed historical data may not have any missing values, even in a scenario where the historical data has certain missing values. In an embodiment, a GCM distribution determined from the original historical data may be same as a GCM distribution determined from the transformed historical data. As the transformed multivariate dataset does not include any missing values or categorical data, the GCM distribution determined from the transformed historical data may be more accurate in identifying the one or more clusters in the historical data than the GCM distribution determined from the original historical data, which may have missing values or categorical data.

For example, the historical data includes a physiological parameter such as gender, which is of a categorical data type. Thus, observations for the physiological parameter “gender” may have either a value of “Male” or “Female”, which may in turn be represented as “0” and “1” in the historical data. In an embodiment, the processor 202 may determine a binomial distribution of the observations of gender in the historical data. Thereafter, the processor 202 may fit the binomial distribution to a GMM distribution based on the rank transformation. Thus, the observations of categorical data type in the historical data may be converted into numerical data in the transformed historical data.

Further, in case of a missing value y_{i,j} in the historical data, the value of the latent variable z_{i,j} may be imputed from an unconstrained mixture of normal distributions (i.e., a GMM) with parameters Θ (which are same as the one or more parameters (Θ) of the first distribution) during re-sampling of the latent variable Z (as discussed in the steps 310 through 318). Thus, the transformed historical data, represented in terms of the latent variable, may not have any missing values.

At step 306, the latent variable is determined based on an inverse cumulative distribution of the transformed historical data. In an embodiment, the processor 202 is configured to determine the latent variable based on the inverse cumulative distribution of the transformed historical data using the following equation:

Z=φ⁻¹(Y_{R}) (4)

where,

Z: the latent variable of the GCM distribution (refer equation 1);

Y_{R}: the transformed historical data; and

φ⁻¹: an inverse cumulative distribution function.

At step 308, the one or more parameters of the first distribution associated with the one or more health conditions in the historical data are estimated. In an embodiment, processor 202 is configured to estimate the one or more parameters of the first distribution based on the latent variable. In a scenario where the first distribution corresponds to the GCM distribution, the one or more parameters (denoted by Θ) may include at least one of a mean (denoted by μ_{g}), a covariance matrix (denoted by Θ_{g}), and a mixing proportion (denoted by π_{g}), of a cluster component (denoted by g) associated with the first distribution. Thus, for a cluster component, g (from the one or more cluster components, G), the one or more parameters may be represented as Θ=[μ_{g}, Σ_{g}, π_{g}]. In an embodiment, the processor 202 may estimate the one or more parameters of the first distribution using a Gibbs sampling technique or an Expectation Maximization (EM) technique. For example, the processor 202 may determine the one or more parameters of the GCM distribution by maximizing the extended rank likelihood function P (Z∈D|Θ) (determined using equation 3) as a function of Θ using an EM technique or a Bayesian technique. Alternatively, the processor 202 may use a Gibbs sampling technique to obtain a Bayesian inference estimate for the one or more parameters of the GCM by constructing a Markov chain having a stationary posterior distribution equal to: P(Θ|Z∈D)∝P(Θ)∝P(Z∈D|Θ).

A person skilled in the art would appreciate that the scope of the disclosure is not limited to determining the one or more parameters of the first distribution, as disclosed above. The one or more parameters may be determined using any statistical technique known in the art without departing from the scope of the disclosure.

After determining the one or more parameters of the first distribution, the processor 202 may re-sample the latent variable (i.e., Z) for each physiological parameter, as described in steps 310 through 318.

At step 310, a lower bound and an upper bound of the latent variable for a physiological parameter from the one or more physiological parameters is determined. In an embodiment, the processor 202 is configured to determine the lower bound (denoted by Zₗ) and the upper bound (denoted by Zᵤ) of the latent variable Z for the j^{th} physiological parameter using the following equations:

Zₗ=max{zᵢⱼ: yᵢⱼ<y} (5)

Zᵤ=min{zᵢⱼ: yᵢⱼ>y} (6)

where,

Zₗ: the lower bound of the latent variable Z for the j^{th} physiological parameter;

Zᵤ: the upper bound of the latent variable Z for the j^{th} physiological parameter;

y: each unique observation in the historical data, for a given value of the j^{th} physiological parameter; and

yᵢⱼ:i^{th} observation of the j^{th} physiological parameter in the historical data.

In an embodiment, the processor 202 may utilize the comparator 210 to perform the comparisons involved in the equations 5 and 6. For instance, the processor 202 may use the comparator 210 to compare a given value of yᵢⱼ with y (i.e., each unique value of yᵢⱼ, for the j^{th} physiological parameter).

At step 312, a second distribution of the physiological parameter is determined. In an embodiment, the processor 202 is configured to determine the second distribution of the physiological parameter, associated with each of the one or more health conditions in the historical data (i.e., the G clusters in the first distribution) based on the one or more parameters of the first distribution. In a scenario where the first distribution corresponds to the GCM distribution, in an embodiment, the processor 202 may first determine a GMM distribution for the physiological parameter based on the one or more parameters of the GCM distribution (determined at step 308). To determine the GMM distribution, the processor 202 may determine one or more parameters of the GMM distribution based on the one or more parameters of the GCM distribution. For example, for the j^{th} physiological parameter, the processor 202 may determine a mean μ_{gj} and a standard deviation σ_{gj}, for each cluster g of the GMM distribution, based on the value of a mean μ_{gj} and a covariance matrix Σ_{gj} for the respective cluster g of the GCM distribution. After determining the GMM distribution for the physiological parameter, the processor 202 may determine the second distribution by truncating each cluster g (e.g., a Gaussian/Normal distribution) in the GMM based the lower bound (i.e., Zₗ) and the upper bound (i.e., Zᵤ) of the latent variable Z for the physiological parameter (determined at step 310). In an embodiment, the second distribution may be represented by the following expression:

TN(μ_{gj}, σ_{gj}, Zₗ, Zᵤ), for g=1, 2, 3 . . . G (7)

where,

μ_{gj}: Mean of the Gaussian distribution from the g^{th} cluster component of the GMM for the j^{th} dimension;

σ_{gj}: Standard deviation of the Gaussian distribution from the g^{th} cluster component of the GMM for the jth dimension; and

TN: Truncated Normal distribution formed by truncation of the Gaussian distribution from the g^{th} cluster component of the GMM based on the lower bound (Zₗ) and the upper bound (Zᵤ) of the latent variable Z.

At step 314, a random variable is sampled from the second distribution of the physiological parameter. In an embodiment, the processor 202 is configured to sample the random variable from the second distribution of the physiological parameter. For each observation yᵢⱼ of the j^{th} physiological parameter in the historical data, the processor 202 may sample the random variable (denoted by R_{gij}) from the second distribution, TN(μ_{gj}, σ_{gj}, Zₗ, Zᵤ), for each cluster g=1, 2, . . . G in the GMM of the second distribution. A person skilled in the art would appreciate that any statistical technique known in the art may be used to perform the sampling of the random variable from the second distribution without departing from the spirit of the disclosure.

At step 316, the latent variable is updated based on the random variable. In an embodiment, the processor 202 is configured to update the latent variable based on the random variable sampled at step 314. In an embodiment, the updating of the latent variable may also be based on the mixing proportion of the one or more cluster components (i.e., π_{g}) in the first distribution (i.e. the one or more health conditions in the historical data). In an embodiment, the processor 202 may perform the updating of the latent variable Z using the following equation:

Zᵢⱼ=Θ_{g=1}^{G}π_{g}R_{gij}, f or each i (8)

Zᵢⱼ: the value of the latent variable for the i^{th} observation of the j^{th} physiological parameter in the historical data;

π_{g}: the mixing proportion of the cluster component g (i.e., the g^{th} health condition in the historical data) of the first distribution; and

Rgij: the value of the random variable sampled from the g^{th} cluster component in the GMM of the second distribution for the i^{th} observation of the j^{th} physiological parameter in the historical data.

A person skilled in the art would appreciate that the truncation of each cluster g (e.g., a Gaussian/Normal distribution) in the GMM of the second distribution based on the lower bound (Zₗ) and the upper bound (Zᵤ) of the latent variable Z (at step 312) may ensure that the values of the latent variable updated at step 316 lie within the set D (represented in expression 2).

At step 318, a check is performed to determine whether all physiological parameters in the historical data have been processed. In an embodiment, the processor 202 is configured to perform the check. The processor 202 performs an iteration of the steps 310 through 318 for each physiological parameter, not yet been processed. Alternatively, if the processor 202 determines that all the physiological parameters have been processed, the processor 202 performs step 320.

At step 320, a check is performed to determine whether a termination condition is reached. In an embodiment, the processor 202 is configured to perform the check. Based on the check, if it is determined that the termination is reached, the processor 202 may perform step 324. Otherwise, the processor 202 performs an iteration of step 322 followed by the steps 310 through 320. In an embodiment, the termination condition may correspond to performing a predetermined number of iterations of the step 322 followed by the steps 310 through 320. Alternatively, when the values of the updated latent variables in two consecutive iterations are approximately equal or differ by a small threshold value, the processor 202 may determine that the value of the latent variable has converged to a final value and the termination condition has been reached.

A person skilled in the art would appreciate that the scope of the disclosure is not limited to the terminal condition, as discussed above. The disclosure may be implemented with any terminal condition without departing from the scope of the disclosure.

At step 322, the one or more parameters of the first distribution are re-estimated based on the updated latent variable. In an embodiment, if the processor 202 determines at step 320 that the termination condition has not been reached, the processor 202 is configured to re-estimate the one or more parameters of the first distribution based on the updated value of the latent variable at step 316. In an embodiment, the one or more parameters may be re-estimated in a manner similar to the estimation of the one or more parameters described in step 308, by using the updated value of the latent variable.

At step 324, the one or more health conditions are identified in the historical data by utilizing the first distribution. In an embodiment, if the processor 202 determines at step 320 that the termination condition has been reached, the processor 202 is configured to use the first distribution to identify the one or more health conditions in the historical data. In an embodiment, the processor 202 may determine the first distribution based on the updated value of the latent variable and the updated one or more parameters associated with the first distribution. Further, the processor 202 may assign the final values of the latent variable as labels for the each of the one or more health conditions in the historical data. Thus, the medical records of each of the one or more second human subjects (i.e., the observations) in the historical data are clustered into the one or more health conditions, based on the final value of the latent variable in the first distribution. For example, the processor 202 labels an observation y, in the historical data with a latent variable of value zᵢ=z*. In such a scenario, the processor 202 may use the value of zi=z* for the observation yi, to identify the health condition (e.g., a g^{th} cluster component from the G cluster components of the first distribution) in which the observation yᵢ has been categorized.

At step 326, a classifier is trained based on the first distribution. In an embodiment, the processor 202 is configured to train the classifier. As discussed above, the processor 202 may determine the first distribution based on the updated one or more parameters and the updated latent variable. In an embodiment, the processor 202 may train the classifier based on the first distribution and the historical data, using one or more machine learning techniques known in the art. Examples of the classifier may include, but are not limited to, a Support Vector Machine (SVM), a Logistic Regression, a Bayesian Classifier, a Decision Tree Classifier, a Copula-based Classifier, a K-Nearest Neighbors (KNN) Classifier, or a Random Forest (RF) Classifier.

A person skilled in the art would appreciate that the scope of the disclosure is not limited to the training of the classifier, as discussed above. The classifier may be trained using any machine learning or artificial intelligence technique known in the art without departing from the spirit of the disclosure.

At step 328, a measure of the one or more physiological parameters of the first human subject is received. In an embodiment, the processor 202 is configured to receive the measure of the one or more physiological parameters of the first human subject from the human subject-computing device 106 of the first human subject. In an embodiment, as discussed, the one or more biosensors, for example, 108a, may be inbuilt within the human subject-computing device 106. Alternatively, the one or more biosensors, for example, 108a may be coupled to the human subject-computing device 106 through the one or more DAQ interfaces, for example, 110a. In an embodiment, the one or more biosensors, for example, 108a, may measure the one or more physiological parameters of the first human subject. Thereafter, the human subject-computing device 106 may send the one or more physiological parameters of the first human subject to the processor 202.

At step 330, the health condition of the first human subject is predicted using the classifier. In an embodiment, the processor 202 is configured to predict the health condition of the first human subject using the classifier. Prior to predicting the health condition, the processor 202 may receive a measure of the one or more physiological parameters of the first human subject from the user. Based on the one or more physiological parameters of the first human subject, the processor 202 may predict the health condition of the first human subject by utilizing the classifier. Further, the processor 202 may display the predicted health condition of the first human subject through a user-interface on the human subject-computing device 106 of the first human subject. In an embodiment, the health condition may correspond to at least one of a disease risk, a disease symptom, an onset of a disease, a recovery from a disease, or an effect of medications for a disease.

A person having ordinary skill in the art would understand that the scope of the disclosure should not be limited to determining a health condition of a human subject. In an embodiment, similar medical data may be analyzed to draw out various inferences. For instance, insurance data pertaining to health care may be analyzed to determine health insurance frauds.

Further, the method described in flowchart 300 may be applied at various levels in the healthcare industry such as at individual patient level through analysis of Electronic Medical Records (EMR), or at hospital level (e.g., identifying a group of patients having risk of getting involved in health insurance frauds). For example, the historical data may correspond to a multivariate dataset including medical insurance records of one or more individuals. In such a scenario, the p-dimensional variable in each medical insurance record may correspond to one or more insurance related parameters such as age of an insured person, one or more physiological parameters of the insured person, premium being paid by the insured person, insurance amount, coverage limit, and so on. Thus, the process described in the flowchart 300 may be utilized to determine insurance frauds, recommend insurance amounts, etc.

Further, a person skilled in the art would appreciate that the scope of the disclosure should not be limited to predicting the health condition of the first human subject. In an embodiment, the disclosure may be implemented for identifying one or more categories in any multivariate dataset. Further, the disclosure may be implemented for predicting a category from the one or more categories into which a new record of the multivariate dataset may classified. For example, the disclosure may be implemented to analyze a financial dataset to determine a credit risk category of a customer. Further, the financial dataset may be analysed to categorize the customers in one or more categories of buying behaviors. The financial dataset may include various types of financial data such as, but not limited to, loan risk assessment data, insurance data, bank statements, and bank transaction data.

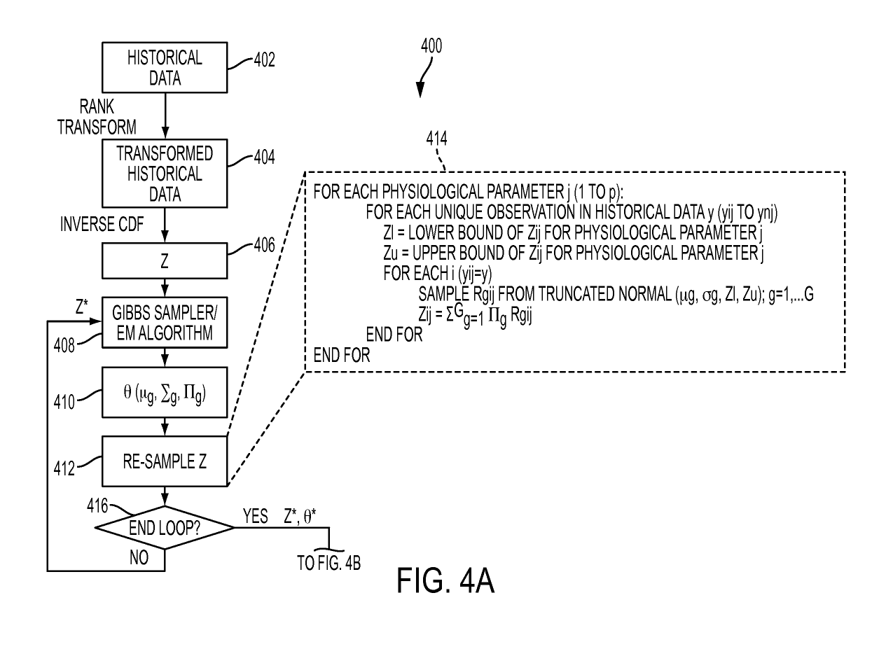

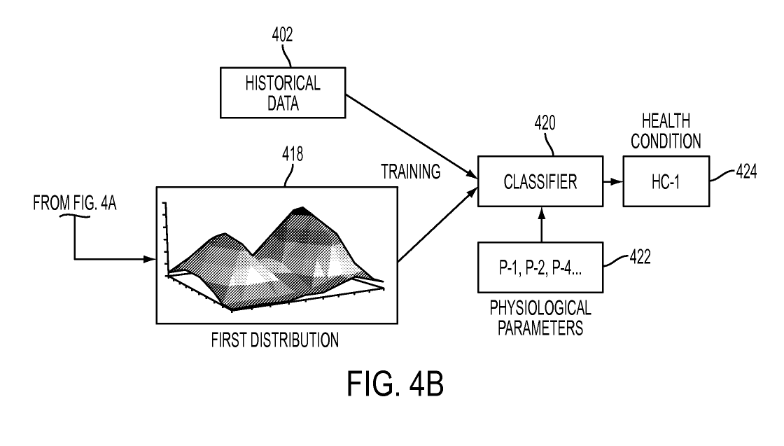

FIGS. 4A and 4B illustrate a flow diagram 400 of method for predicting the health condition of the first human subject, in accordance with at least one embodiment. The flow diagram 400 has been described in conjunction with FIG. 1, FIG. 2, FIG. 3A, and FIG. 3B.

The processor 202 receives the historical data including the medical records of the one or more second human subjects (depicted by 402). In an embodiment, the processor 202 may retrieve the historical data (depicted by 402) from a database or receive the historical data (depicted by 402) from the user, as described in step 302. Further, in an embodiment, the processor 202 may receive a user-input pertaining to a number of the one or more health conditions (denoted by G clusters) in the historical data. Thereafter, the processor 202 may apply the rank transformation on the historical data (depicted by 402) to obtain the transformed historical data (depicted by 404), in manner similar to that disclosed in step 304. Further, the processor 202 determines the latent variable Z (depicted by 406) based on the inverse cumulative distribution of the transformed historical data (depicted by 404), in manner similar to that disclosed in step 306. Thereafter, the processor 202 may estimate the one or more parameters of the first distribution (i.e., Θ=[μ_{g}, Σ_{g}, π_{g}], depicted by 410) using a Gibbs Sampler/EM Algorithm (depicted by 408 ), in a manner similar to that discussed in 308.

After estimating the one or more parameters of the first distribution (depicted by 410), in an embodiment, the processor 202 may resample the latent variable Z (depicted by 412), in a manner similar to that described in the steps 310 through 318. A pseudo-code 414 illustrates the resampling of the latent variable Z in detail. The pseudo-code 414 is represented as under:

1. For each physiological parameter j (1 to p):

2. For each unique observation in historical data y (yⱼ to yₙⱼ):

3. Zₗ=Lower bound of Zᵢⱼ for physiological parameter j

4. Zᵤ=Upper bound of Zᵢⱼ for physiological parameter j

5. for each i (yij=y):

6. Sample random variable R_{gij} from Truncated Normal (μ_{g}, σ_{g}, Zₗ, Zᵤ); g=1, . . . G

7. Zᵢⱼ=Σ_{g=1}^{G}π_{g} R_{gij}

8. end for

9. end for

The determination of the lower and the upper bounds of Zᵢⱼ (i.e., Zₗ and Zᵤ, respectively) in lines 3 and 4, respectively, of the pseudo-code 414 has been explained in step 310. The determination of the Truncated Normal Distribution (i.e., the second distribution) for the j^{th} physiological parameter has been explained in step 312. Further, the sampling of the random variable R_{gij} from the Truncated Normal distribution (i.e., line 6 of the pseudo-code 414) has been explained in step 314. The updating of the latent variable Z based on the sampled random variable R_{gij} (i.e., line 7 of the pseudo-code 414 ) has been explained in step 316.

After the resampling of the latent variable Z (depicted by 412 and illustrated in detail in the pseudo-code 414 ), the processor 202 may check whether a termination condition for an end of a Gibbs Sampling loop (depicted by 410 through 412) has been reached (depicted by 416). The checking of the termination condition has been explained further in the step 320. If the processor 202 determines that the termination condition of the loop has not been reached, the processor 202 may continue with another iteration of the Gibbs Sampling loop (depicted by 410 through 412) with the updated value of the latent variable sampled at step 412 (depicted by Z*). Thus, the processor 202 may provide the Gibbs Sampler/EM Algorithm (depicted by 408) with the updated latent variable Z* and the Gibbs Sampling loop (depicted by 410 through 412) may be iterated. On the other hand, if the processor 202 determines that the termination condition has been reached, the processor 202 may use the updated latent variable (depicted by Z*) and the final value of the one or more parameters of the first distribution (depicted by Θ*) to identify the one or more health conditions (i.e., the one or more clusters) in the historical data 402, as explained in step 324. In an embodiment, the processor 202 may label the one or more health conditions based on the latent variable value Z*. In an embodiment, the processor 202 may identify the one or more health conditions in the historical data 402 based on the first distribution (depicted by 418).

Thereafter, the processor 202 may train the classifier (depicted by 420) based on the first distribution (depicted by 418) and the historical data 402 using one or more machine learning techniques known in the art, as explained in the step 326. Further, the processor 202 may receive a measure of the one or more physiological parameters (such as, physiological parameters P-1, P-2, P-3, . . . depicted by 422) of the first human subject from the human subject-computing device 106, as explained in step 328. The processor 202 may use the classifier (depicted by 420) to predict the health condition (e.g., the health condition HC-1, depicted by 424) of the first human subject based on the one or more physiological parameters (depicted by 422) of the first human subject, as explained in step 330.

The disclosed embodiments encompass numerous advantages. The disclosure leads to an effective clustering of a multivariate dataset using a GCM distribution. For example, the multivariate dataset may be a healthcare dataset that includes medical records of one or more human subjects. By using the GCM distribution, one or more clusters indicative of one or more health conditions of the one or more human subjects may be identified. The GCM distribution, though a very robust statistical method for clustering data of a numerical data type, may be inefficient while handling data of a categorical data type. Further, the GCM distribution may not perform well in case of missing values in the multivariate dataset. The clustering performance of the GCM distribution may deteriorate further when the multivariate dataset is of a higher dimension (e.g., a dimension greater than 15). The disclosure overcomes the aforementioned shortcomings of the GCM distribution for clustering the multivariate dataset and determination of complex dependencies within the multivariate dataset.

The disclosed methods and systems, as illustrated in the ongoing description or any of its components, may be embodied in the form of a computer system. Typical examples of a computer system include a general-purpose computer, a programmed microprocessor, a micro-controller, a peripheral integrated circuit element, and other devices or arrangements of devices that are capable of implementing the steps that constitute the method of the disclosure.

The computer system comprises a computer, an input device, a display unit and the Internet. The computer further comprises a microprocessor. The microprocessor is connected to a communication bus. The computer also includes a memory. The memory may be Random Access Memory (RAM) or Read Only Memory (ROM). The computer system further comprises a storage device, which may be a hard-disk drive or a removable storage drive, such as, a floppy-disk drive, optical-disk drive, and the like. The storage device may also be a means for loading computer programs or other instructions into the computer system. The computer system also includes a communication unit. The communication unit allows the computer to connect to other databases and the Internet through an input/output (I/O) interface, allowing the transfer as well as reception of data from other sources. The communication unit may include a modem, an Ethernet card, or other similar devices, which enable the computer system to connect to databases and networks, such as, LAN, MAN, WAN, and the Internet. The computer system facilitates input from a user through input devices accessible to the system through an I/O interface.

In order to process input data, the computer system executes a set of instructions that are stored in one or more storage elements. The storage elements may also hold data or other information, as desired. The storage element may be in the form of an information source or a physical memory element present in the processing machine.

The programmable or computer-readable instructions may include various commands that instruct the processing machine to perform specific tasks, such as steps that constitute the method of the disclosure. The systems and methods described can also be implemented using only software programming or using only hardware or by a varying combination of the two techniques. The disclosure is independent of the programming language and the operating system used in the computers. The instructions for the disclosure can be written in all programming languages including, but not limited to, “C,” “C++,” “Visual C++” and “Visual Basic.” Further, the software may be in the form of a collection of separate programs, a program module containing a larger program or a portion of a program module, as discussed in the ongoing description. The software may also include modular programming in the form of object-oriented programming. The processing of input data by the processing machine may be in response to user commands, the results of previous processing, or from a request made by another processing machine. The disclosure can also be implemented in various operating systems and platforms including, but not limited to, “Unix,” “DOS,” “Android,” “Symbian,” and “Linux.”

The programmable instructions can be stored and transmitted on a computer-readable medium. The disclosure can also be embodied in a computer program product comprising a computer-readable medium, or with any product capable of implementing the above methods and systems, or the numerous possible variations thereof.

Various embodiments of methods and systems for predicting health condition of a human subject have been disclosed. However, it should be apparent to those skilled in the art that modifications in addition to those described, are possible without departing from the inventive concepts herein. The embodiments, therefore, are not restrictive, except in the spirit of the disclosure. Moreover, in interpreting the disclosure, all terms should be understood in the broadest possible manner consistent with the context. In particular, the terms “comprises” and “comprising” should be interpreted as referring to elements, components, or steps, in a non-exclusive manner, indicating that the referenced elements, components, or steps may be present, or utilized, or combined with other elements, components, or steps that are not expressly referenced.

A person having ordinary skills in the art will appreciate that the system, modules, and sub-modules have been illustrated and explained to serve as examples and should not be considered limiting in any manner. It will be further appreciated that the variants of the above disclosed system elements, or modules and other features and functions, or alternatives thereof, may be combined to create other different systems or applications.

Those skilled in the art will appreciate that any of the aforementioned steps and/or system modules may be suitably replaced, reordered, or removed, and additional steps and/or system modules may be inserted, depending on the needs of a particular application. In addition, the systems of the aforementioned embodiments may be implemented using a wide variety of suitable processes and system modules and is not limited to any particular computer hardware, software, middleware, firmware, microcode, or the like.

The claims can encompass embodiments for hardware, software, or a combination thereof.

It will be appreciated that variants of the above disclosed, and other features and functions or alternatives thereof, may be combined into many other different systems or applications. Presently unforeseen or unanticipated alternatives, modifications, variations, or improvements therein may be subsequently made by those skilled in the art, which are also intended to be encompassed by the following claims.

What is claimed is:

1. A method for predicting a health condition of a first human subject, the method comprising:
extracting, by one or more processors, a historical data comprising a measure of one or more physiological parameters associated with each of one or more second human subjects;
determining, by said one or more processors, a latent variable based on an inverse cumulative distribution of a transformed historical data, wherein said transformed historical data is determined by ranking of said historical data;
estimating, by said one or more processors, one or more parameters of a first distribution deterministic of one or more health conditions in said historical data, based on said latent variable;
for each physiological parameter from said one or more physiological parameters:
sampling, by said one or more processors, a random variable from a second distribution of said physiological parameter, based on said one or more parameters;
updating, by said one or more processors, said latent variable based on said random variable;
re-estimating, by said one or more processors, said one or more parameters based on said updated latent variable;
training, by said one or more processors, a classifier based on said first distribution;
receiving, by said one or more processors, a measure of said one or more physiological parameters associated with said first human subject; and
predicting, by said one or more processors, said health condition of said first human subject by utilizing said classifier based on said received measure of said one or more physiological parameters associated with said first human subject.

2. The method of claim 1, wherein said one or more physiological parameters comprise at least one of a blood glucose level, a blood pressure, an age, a cholesterol level, a heart rate, a breath carbon-dioxide concentration, or a breath oxygen concentration.

3. The method of claim 1, wherein said one or more parameters are estimated by utilizing one of a Gibbs sampling technique or an Expectation-Maximization (EM) technique.

4. The method of claim 1, wherein said second distribution is truncated based on a lower bound and an upper bound of said latent variable for said physiological parameter.

5. The method of claim 1, wherein said first distribution corresponds to a Gaussian Copula Mixture distribution.

6. The method of claim 1, wherein said one or more parameters comprise at least one of a mean, a covariance matrix, and a mixing proportion, of a cluster component associated with said first distribution.

7. The method of claim 1 further comprising determining, by said one or more processors, said second distribution of said physiological parameter for each of said one or more health conditions.

8. The method of claim 1, wherein said updating of said latent variable is based on a mixing proportion of each of said one or more health conditions in said historical data.

9. The method of claim 1, wherein said ranking of said historical data corresponds to an extended rank likelihood.

10. The method of claim 1, wherein a data type associated with said historical data corresponds to at least one of a numerical data type or a categorical data type.

11. The method of claim 1, wherein said historical data corresponds to a multivariate dataset from which said one or more health conditions are identifiable based on said first distribution.

12. The method of claim 1, wherein each of said one or more health conditions corresponds to at least one of a disease risk, a disease symptom, an onset of a disease, a recovery from a disease, or an effect of medications for a disease.

13. A system for predicting a health condition of a first human subject, the system comprising:
one or more processors configured to:
extract a historical data comprising a measure of one or more physiological parameters associated with each of one or more second human subjects;
determine a latent variable based on an inverse cumulative distribution of a transformed historical data, wherein said transformed historical data is determined by ranking of said historical data;
estimate one or more parameters of a first distribution deterministic of one or more health conditions in said historical data, based on said latent variable;
for each physiological parameter from said one or more physiological parameters:
sample a random variable from a second distribution of said physiological parameter, based on said one or more parameters;
update said latent variable based on said random variable;
re-estimate said one or more parameters based on said updated latent variable;
train a classifier based on said first distribution;
receive a measure of said one or more physiological parameters associated with said first human subject; and
predict said health condition of said first human subject by utilizing said classifier based on said received measure of said one or more physiological parameters associated with said first human subject.

14. The system of claim 13, wherein said one or more parameters are estimated by utilizing one of a Gibbs sampling technique or an Expectation-Maximization (EM) technique.

15. The system of claim 13, wherein said second distribution is truncated based on a lower bound and an upper bound of said latent variable for said physiological parameter.

16. The system of claim 13, wherein said first distribution corresponds to a Gaussian Copula Mixture distribution.

17. The system of claim 13, wherein said updating of said latent variable is based on a mixing proportion of each of said one or more health conditions in said historical data.

18. The system of claim 13, wherein said ranking of said historical data corresponds to an extended rank likelihood.

19. A computer program product for use with a computing device, the computer program product comprising a non-transitory computer readable medium, wherein the non-transitory computer readable medium stores a computer program code for predicting a health condition of a first human subject, wherein the computer program code is executable by one or more processors in the computing device to:
extract a historical data comprising a measure of one or more physiological parameters associated with each of one or more second human subjects;
determine a latent variable based on an inverse cumulative distribution of a transformed historical data, wherein said transformed historical data is determined by ranking of said historical data;
estimate one or more parameters of a first distribution deterministic of one or more health conditions in said historical data, based on said latent variable;
for each physiological parameter from said one or more physiological parameters:
sample a random variable from a second distribution of said physiological parameter, based on said one or more parameters;
update said latent variable based on said random variable;
re-estimate said one or more parameters based on said updated latent variable; and
train a classifier based on said first distribution;
receive a measure of said one or more physiological parameters associated with said first human subject; and
predict said health condition of said first human subject by utilizing said classifier based on said received measure of said one or more physiological parameters associated with said first human subject.
